(19) United States
(12) Patent Application Publication
(10) Pub. No.: US 2019/0105486 A1
(43) Pub. Date: Apr. 11, 2019

(54) SUPPORT FOR TRANSDERMAL APPLICATION OF SUBSTANCES

(71) Applicant: Elisabetta ROSSI, Missaglia (IT)

(72) Inventor: Guido PADUANO, Missaglia (IT)

(21) Appl. No.: 16/064,260

(22) PCT Filed: Nov. 30, 2016
(86) PCT No.: PCT/IB2016/057177

§ 371 Date: Jun. 20, 2018

(30) Foreign Application Priority Data
Dec. 4, 2015 (IT) 102015000080363

(51) Int. Cl. A61N 1/04

(52) U.S. Cl. CPC A61N 1/0428; A61N 1/048; A61N 1/0476; A61N 1/0412

(57) ABSTRACT

The invention relates to a support with electrodes (20) and a gel containing active ingredients, for transdermal application of the latter. The support consists of a base layer (10) made of flexible material (non-woven fabric, PET, PVC, etc.), whereas the electrodes are made of a conductive material such as carbon, graphene, germanene or the like, which is impressed onto the support by hot or cold pressing, silk-screening, ink jet or the like. The electrodes can be connected to a power supply unit via a USB connector or the like.

In a general aspect, the present invention relates to transdermal application of substances by means of electrophoretic or iontophoretic techniques.

As is known, the latter are techniques that allow substances to pass through the epidermis and/or the cutaneous barrier at ionic-molecular level to reach body parts to be treated. The substances are active principles, such as drugs of various classes, medical devices, integrators, phytotherapeutic extracts, herbal powders and the like, whether alone or combined with other substances, which are delivered by exploiting the currents supplied by the generator.

This substance administration technique has some interesting advantages over the cutaneous technique for application of creams and ointments, the intramuscular injection technique, the oral administration technique, and also those known operator-dependent techniques that make use of manual electrodes, in addition to being non-systemic, non-invasive, free from side effects and, of course, much more effective than simple topical applications.

In fact, as common practice demonstrates, the application of creams and ointments requires that the skin area involved be first smeared with the product and then slightly massaged by the user him/herself or by another person, depending on the body part involved.

This will not ensure a uniform application, and hence a regular dosage of the substances that need to be applied: it is in fact clear that, since these operations are carried out manually by a person, it is not possible to always attain an even application because of too many human-related variables.

On the contrary, application via intramuscular injection ensures accurate dosage, but with systemic, non-topical/regional absorption, and, as everybody knows well from personal experience, injections are often painful or anyway annoying, and not everybody can make them.

Last, it is known that oral administration of active principles, just like intramuscular injection, is often accompanied by undesired side effects, and may therefore be contraindicated for certain subjects, such as children or old people or allergic people. It should also be taken into account that the substances that need to be administered can hardly reach entirely the body region to be treated, since they partly scatter within the body (e.g. in the digestive tract or in the vascular system) before reaching that part: this will inevitably lead to substance dosage and application problems as well as to possible allergic reactions.

Without describing any further the clinical and technical aspects of transdermal substance delivery, in regard to which reference should be made to the wide scientific literature available, in this context it may be useful to point out that the devices taken into consideration by the invention are those of the type described in American patent U.S. Pat. No. 5,658,247 (to Henley) or in the International patent applications WO 00/53256 (by Palti) and WO 02/24274 by the present Applicant.

In particular, this latter International patent application by the present Applicant, who has filed a number of patents in Europe (EP 1318854), the United States (U.S. Pat. No. 7,162,297) and Russia (RU 2262358), describes a method, a device and a related applicator patch for transdermal application of substances, the electrodes of which are powered with a time-modulated voltage to provide improved application efficacy compared to the prior art.

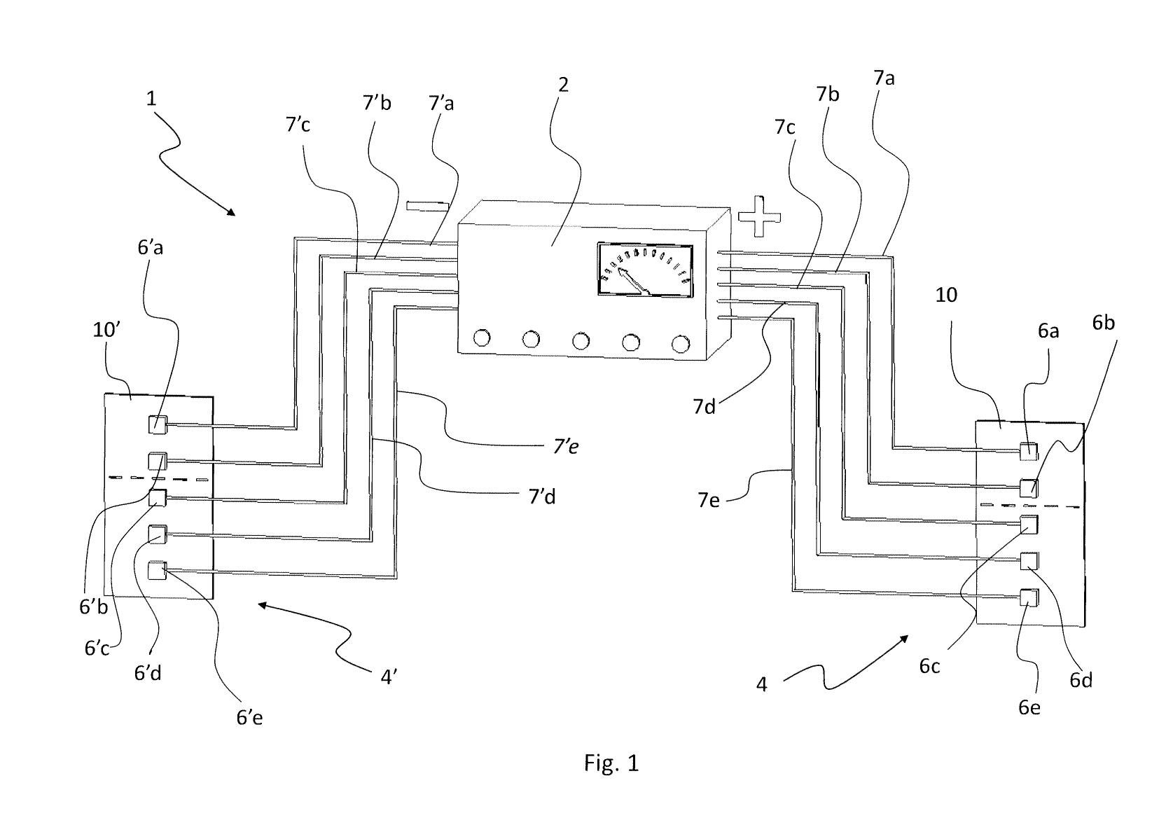

FIG. 1 from International patent application WO 02/24274 shows the apparatus described therein. As can be seen, the electrodes are arranged on the applicator patch, which may be in the form of a closed flexible envelope containing the substance to be administered, or a textile substrate supporting the latter; the electrodes are electrically connected to a power supply apparatus; the latter regulates and controls the electric current and/or voltage supplied to the electrodes according to a pulsed, frequency-modulated and amplitude-modulated trend.

The results attained by this kind of device for transdermal substance delivery have been favourable. Therefore, with a view to gaining better performance, the Applicant has focused the attention to some operational aspects thereof.

For example, one of such aspects relates to delivering substances through the cutaneous barrier to reach deep tissues without damaging the skin and without interacting with the circulatory system.

In fact, when treatments need to be delivered in internal regions of the body at a depth of approx. ten centimeters from the skin area where the electrodes are applied (e.g. the chest, the thighs, the pelvis, etc.), one usually tends to increase the voltage of the electrodes, thus running the risk of damaging the skin where the electrodes have been applied, resulting in burns caused by local current discharges.

Furthermore, the present Applicant has found out that the presence of close electrodes may have some contraindications, such as local overheating of the skin or deviation of the electric fields, which is detrimental to substance penetration.

Finally, common experience with the applicator devices known in the art has revealed a need for an operator to connect all the electrodes, one by one, to the control apparatus by means of conductors or clamps or the like: this allows using disposable applicator patches, which can be disconnected from the machine and disposed of after use, thus ensuring the utmost hygiene.

However, this solution has the drawback that it may require some time when the number of electrodes is large (e.g. 6, 8 or more), or that it may be difficult to connect the electrodes in certain areas of the body whereto the patch has been applied.

This situation does not appear to have been overcome by the other known devices mentioned above (such as a roller or a ball associated with a bottle of conductive gel and active principles), wherein the electrodes, since they are not of the disposable type because they are fixedly connected to the control apparatus, must be sanitized and sterilized before each treatment: this may even take longer than connecting the electrodes, and still it will not ensure a complete elimination of germs and risks of transmission of diseases, because of contact with the skin of different patients.

A technical problem at the basis of the invention is therefore to provide a support for electrodes for transdermal substance delivery, the structural and operational features of which can overcome one or more of the above-mentioned drawbacks of the prior art.

It is worth pointing out that, in this description and in the appended claims, the term “support” must be understood in a broad sense to include both a simple substrate, whether textile or of a different nature (e.g. felt, non-woven fabric, film, etc.), configured as a band, a compress, a patch or the like, or a closed or partially open envelope capable of containing the substances to be administered, in accordance with prior-art teachings (e.g. WO 02/24274).

In this context, it must also be taken into account that the electrode support is preferably a flexible one but, as will become apparent hereafter, the invention is also applicable to supports having a more or less large rigid or semi-rigid part and a flexible or soft part.

The idea that solves the above-mentioned technical problem is to create a support wherein the electrodes are associated with a substrate, an envelope or the like, as previously described, so that all of them can be connected at once to the power supply apparatus, without a single connection being required for each one of them.

Disposable supports can thus be created, which can be readily replaced and easily and quickly installed.

Preferably, the electrodes may have different shapes and/or dimensions, so that they can be easily adapted to various applications, e.g. depending on the region of the body to be treated, the type of substance to be administered, the physical properties (impedance) of a person's tissues, etc.

Furthermore, in accordance with a preferred embodiment of the invention, the electrodes are associated with the support together with a portion of electric conductors for connecting them to the power supply apparatus: this will facilitate the establishment of contact points for the application of terminals, sockets o similar quick-connection means.

The features of the invention are specifically set out in the claims appended to this description.

Such features, as well as the effects and advantages achieved by the invention, will become more apparent in the light of the exemplary embodiment of the invention that will be described below with reference to the annexed drawings, wherein:

FIG. 1 shows a machine according to the prior art;

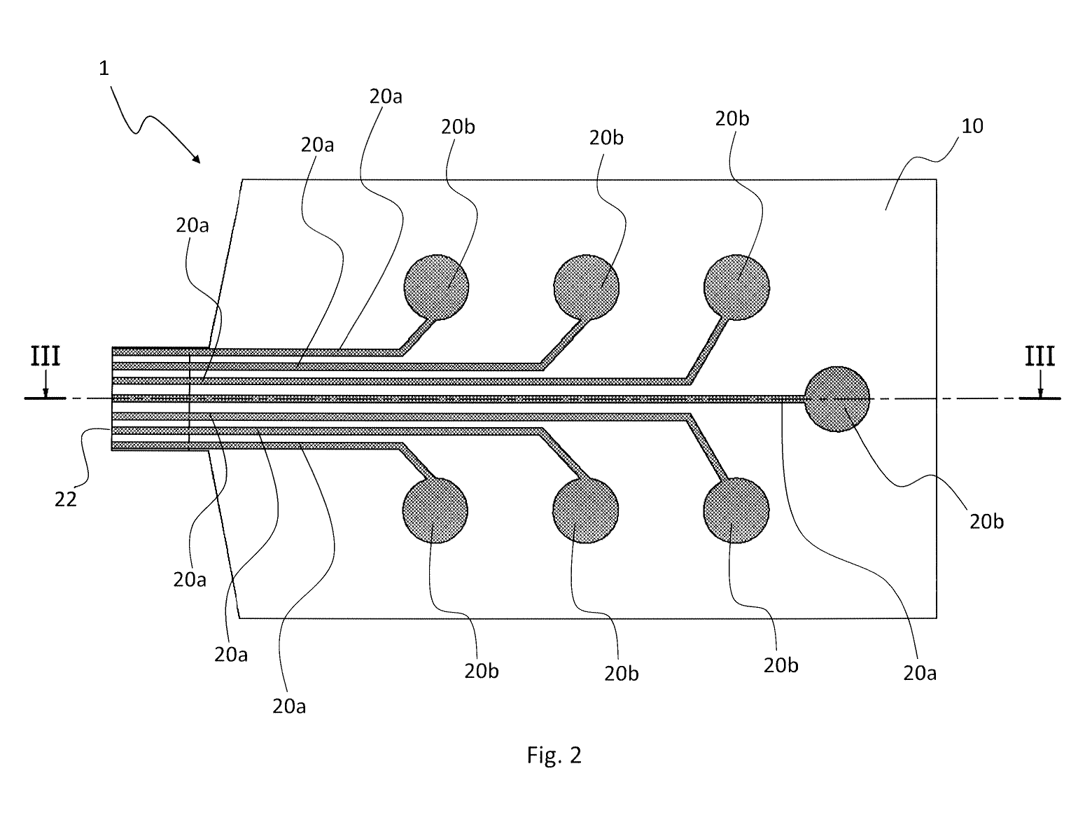

FIG. 2 shows an elevation view of an electrode support according to the invention;

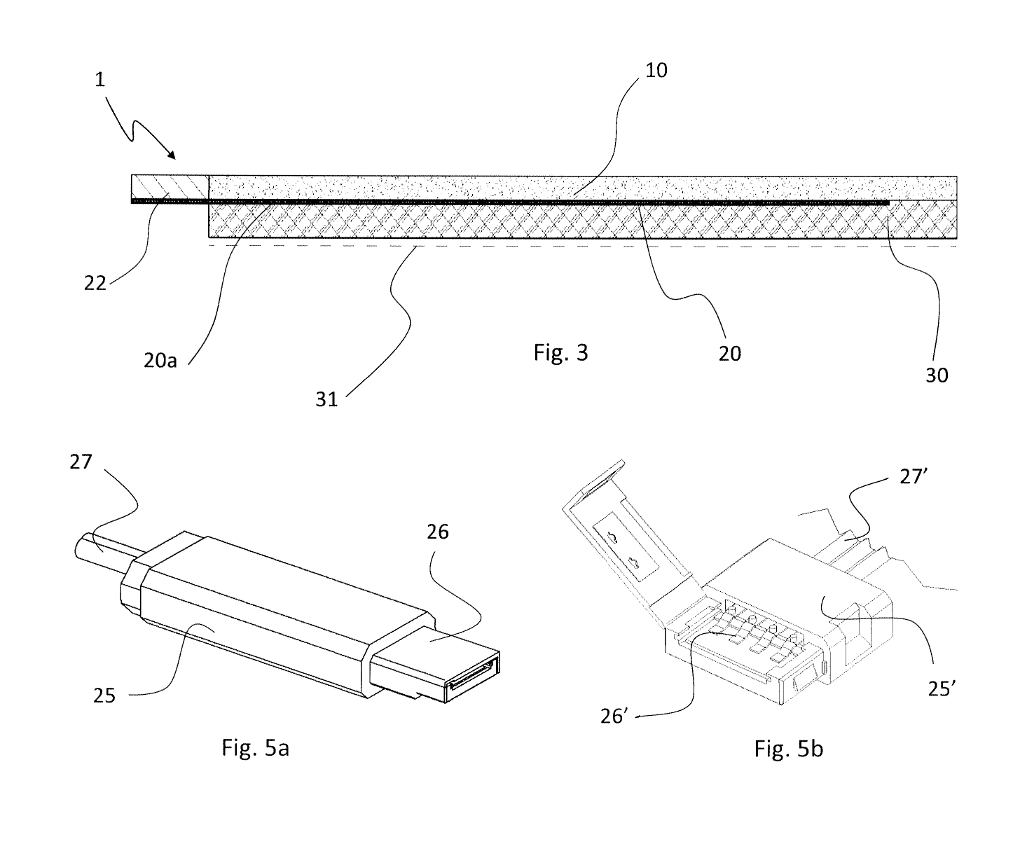

FIG. 3 is a sectional view along line of the support of FIG. 2;

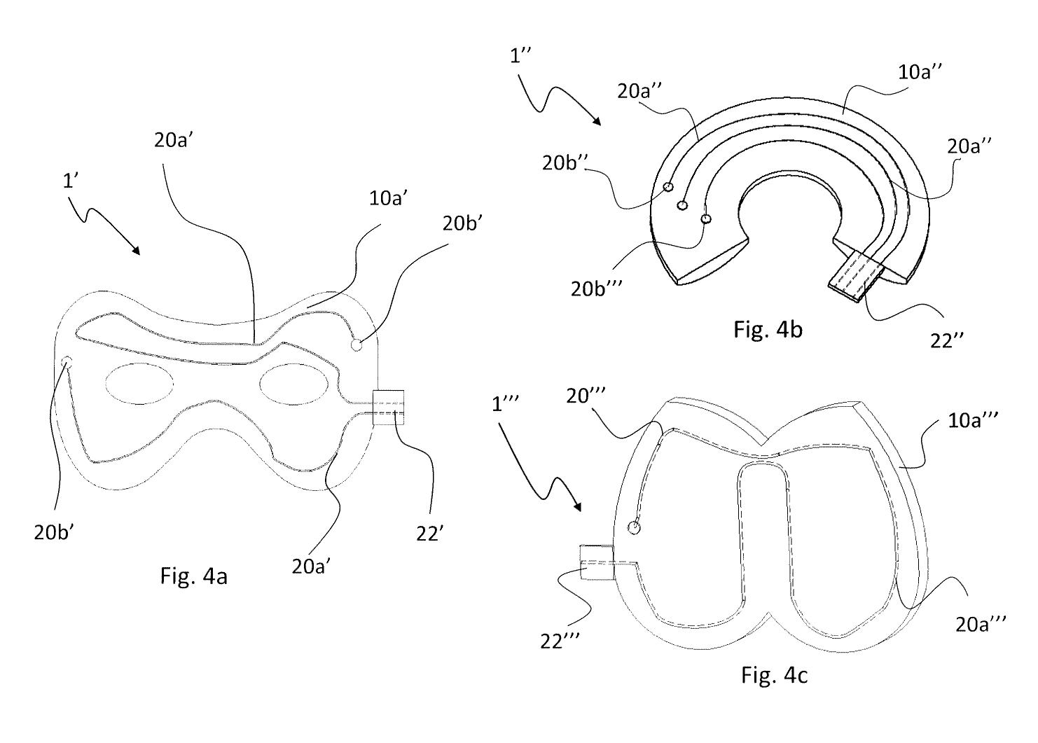

FIGS. 4a, 4b, 4c show respective variants of the support of FIG. 2;

FIGS. 5a, 5b are respective perspective views of electric connectors for the electrode support according to the invention;

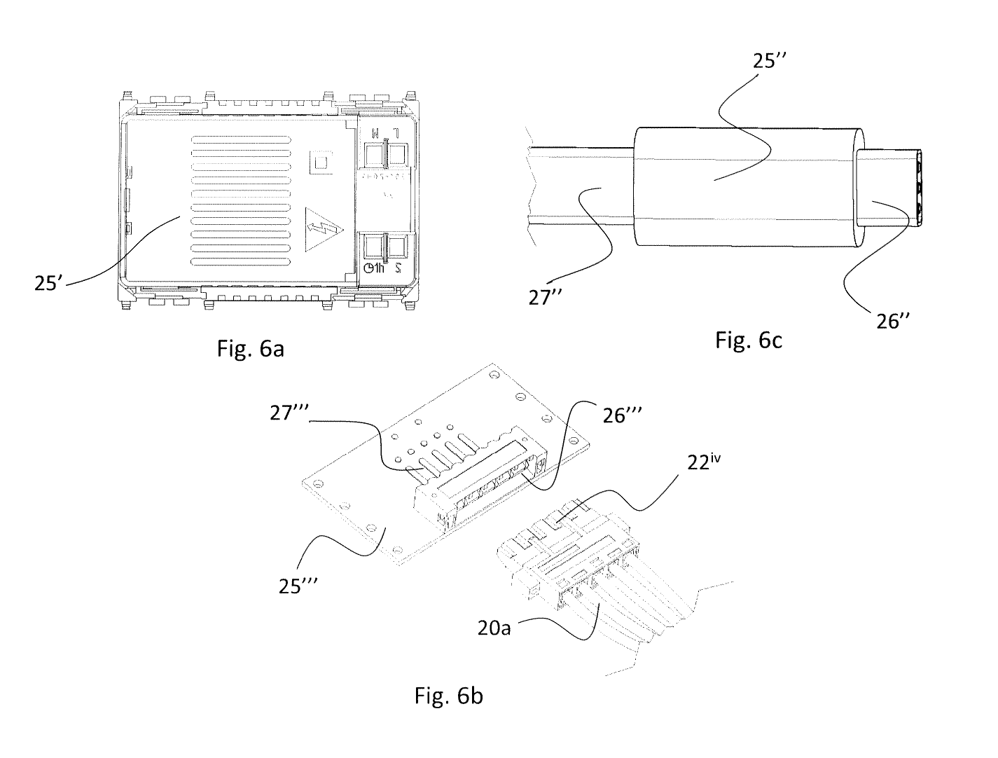

FIGS. 6a, 6b, 6c show respective variants of the connector of FIGS. 5a, 5b;

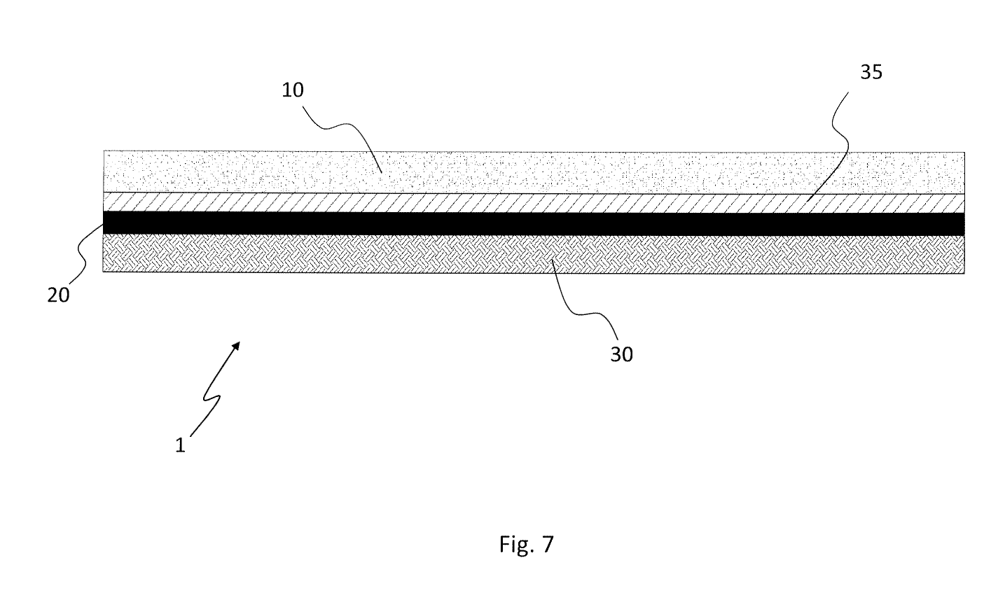

FIG. 7 is a longitudinal sectional view, similar to that of FIG. 2, of a further variant of the support of the invention;

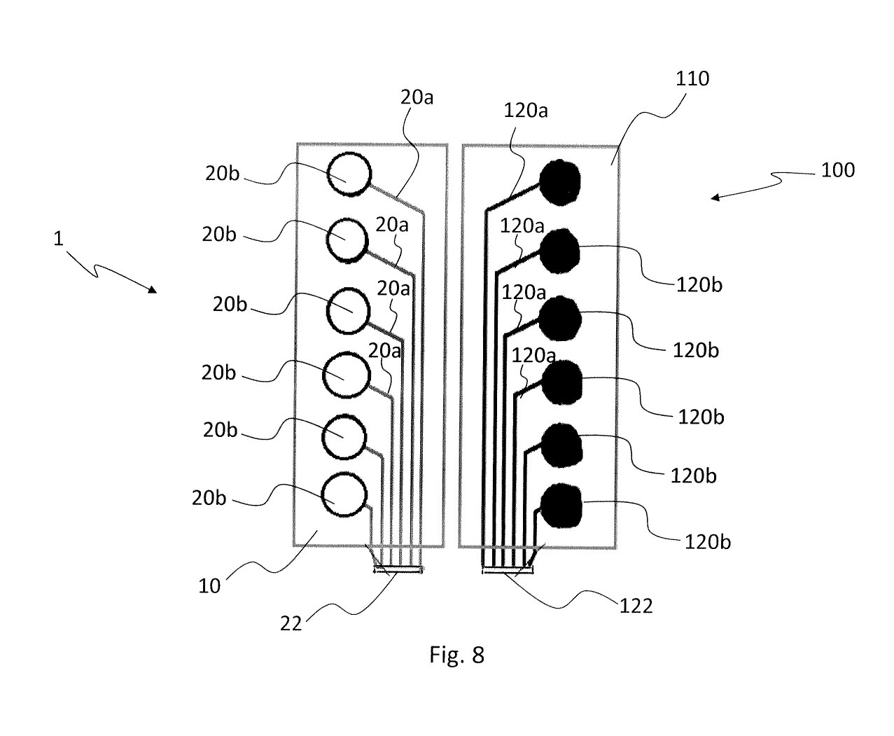

FIG. 8 shows an example of operational use of a pair of supports;

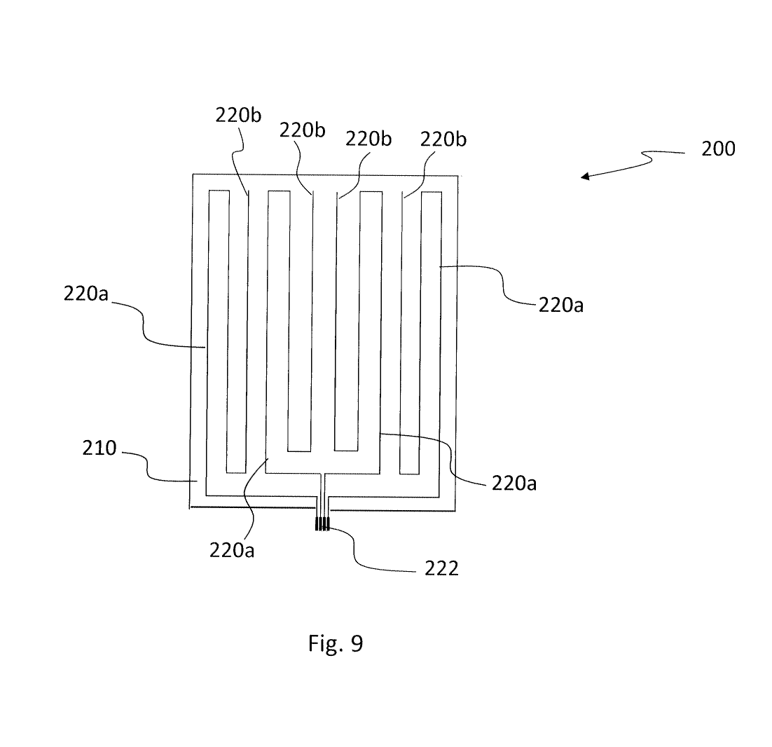

FIG. 9 shows a variant of the support of the invention.

As aforementioned, the first one of the annexed drawings shows an apparatus for transdermal delivery according to the current state of the art; in particular, this machine is the one described in patent application WO 02/24274 by the present Applicant; therefore, for information about the general operation thereof reference should be made to the explanations provided in said application, the contents of which are integrally incorporated in the present description.

Therefore, reference will only be made in this description to the electrode support of the present invention, which can be used both with the apparatus known from WO 02/24274 and with other types of apparatus, as will become apparent hereinafter.

The support in question is indicated as a whole in the drawings by reference numeral 1; it is worth pointing out that the drawings (particularly FIG. 2 and FIG. 9) show, by way of example, two possible embodiments of the support. This should not be considered as a limitation and, as will be better appreciated hereinafter, the support configuration, the number of supported electrodes, the arrangement thereof and other features may vary according to the case.

The support 1 comprises, in this example, a base layer or sheet 10, which is made of flexible material so that it can envelop or anyway be anatomically adapted to the body part whereto substances are to be transdermally applied.

The layer 10 is preferably made of biocompatible material, so that it will not cause any problem neither to the people who have to handle it nor to those on whose skin it has to be applied; in fact, it must be anti-allergic, easily applicable, light, and also pleasant to the touch (i.e. not rough).

The layer 10 can be obtained from natural materials, such as fabric of cotton, flax or other yarns commonly used for sanitary applications (e.g. gauze yarn), or non-woven fabric (NWF, PVC, PET), felt, and also other natural materials, such as leather, hide or the like.

The base layer 10 may also be made, whether wholly or partially, from an artificial material, e.g. in the form of fabric or non-woven fabric or felt, or in the form of a plastic film; materials suitable for this purpose are typically those biocompatible materials commonly used for sanitary purposes, such as polyester, PVC (polyvinyl chloride), polyurethane, polyethylene, silicone, latex or the like.

It must also be pointed out that mixed materials may be used for the base layer 10, such as, for example, elasticized fabric with mixed natural and synthetic fibers, textile layers, felts, non-woven fabrics (NWF), whether impregnated or coated, at least partly, with substances suitable for the purposes that will become apparent below. Also the thickness of the base layer 10 may vary as a function of the materials it is made of In fact, it can be easily understood that the thickness of the layer affects the properties of the latter, such as flexibility and resistance to mechanical stresses (traction, shearing, bending), deformability, elasticity, weight, etc.

Therefore, in the case of textile materials, felts, NWFs and the like, the thickness of the base layer 10 is preferably in the range of 0.3 to 2.0 mm, with surprisingly favourable results obtained with values comprised between 0.5 and 1.5 mm. For such values, optimal adaptability and flexibility of the support 1 has been verified, as well as good compatibility between the base layer 10 and the electrodes 20 applied thereon, as will be further explained below.

The base layer 10 may consist of either a single sheet or a composite structure, with laminated layers joined together; for example, this is the case of a composite base layer comprising a sheet made of natural material (e.g. leather or fabric), whereon a thin polyurethane membrane is applied.

The laminated layers can be joined by stitching or by interposing suitable adhesives or glues, in the form of a continuous film or discrete spots.

Regardless of the way in which the base layer 10 is made, the electrodes 20 are arranged thereon.

The number and size of the latter may differ from case to case, depending on the applications and/or purposes they are intended for. Therefore, there may be cases wherein the support 1 of the invention comprises only one big electrode 20, and cases wherein the support 1 comprises a plurality of smaller electrodes 20, as shown in FIG. 2.

According to a preferred embodiment, the electrodes 20 comprise a linear portion 20a configured substantially like a track of an electric conductor, which extends up to a terminal portion 20b, which may have either the same size or a widened configuration with a larger area.

Preferably, both portions 20a, 20b contribute to the transdermal delivery of the substances, although such delivery will be distributed differently according to the various possible configurations: in other words, one important aspect of the invention is the fact that it allows making electrodes having all kinds of shapes (as will be further illustrated below) to exploit at best their properties and effects.

This will ensure that even the deepest tissues under the skin will be effectively reached, without the need to apply excessive voltage levels that may cause local cutaneous burns.

Thus, for example, by integrating the linear portion 20a into the support 10 it will be possible to obtain that also the portion 20a, which provides a connection to the power supply, will also work as an electrode, thus contributing to transdermal delivery in addition to the terminal part 20b of the electrodes 20.

At any rate, the terminal part is useful for concentrating the current flow locally, thereby avoiding any superficial dissipation that would prevent reaching the deep layers.

By changing the superficial extensions of the portions 20a and 20b it is possible to obtain a different distribution of the electric field afferent thereto, and hence of the corresponding desired effect of the substances being administered.

This effect is further promoted, in the preferred embodiment of the invention, by the fact that the electrodes 20 or, even better, pairs of electrodes, are powered sequentially one after the other, reminding that each electrode is not activated individually but works in pair with a corresponding electrode having the opposite sign, whether a single support or two separate supports are used, in that the electric charge must necessarily be + on one side and − on the other side (and vice versa at the end of every impulse cycle), with a time offset of approx. 1-5 seconds. Advantageously, the electric signals are modulated ones, preferably sine wave signals or the like: this will prevent any interference between the electric currents associated with each electrode 20.

Therefore, it can be easily understood that the connection portion 20a of the latter can be easily configured with a straight, curved, broken or mixed shape, so as to adapt it to the anatomical shape of the body, to the dimensions of the support 10 and/or to the treatment to be carried out.

The size of the electrodes 20, inclusive of both the linear portion 20a and the terminal portion 20b, is preferably in the range of 0.5 to 3 cm². In particular, the Applicant has attained surprisingly good results with a ratio between the terminal portion 20b and the linear portion 20a substantially unitary or anyway lower than 2-2.5.

In general it can be stated that, all other conditions being equal (i.e. power supply current and/or voltage, substance to be delivered, patient, type of treatment, etc.), the electrodes 20 may be smaller than those known in the art, since they are more efficient.

This is a corollary to the fact that, in the preferred embodiment of the invention, the support 1 also includes a part of the conductors 20a that connect the terminals 20b of the electrodes 20 to the power supply unit (not shown in the drawings because per se known, e.g. from WO 02/24274).

In other words, by applying onto the base layer 10 also the portion 20a that connects the terminal portion 20b of the electrodes 20 to the power supply connection 22, it is possible to create connections without solution of continuity that will eliminate any contact resistance, which is present between metal electrodes and conductors in the prior art, thereby improving the overall energetic efficiency of the system. Furthermore, according to a preferred solution the linear portions 20a and the terminal portions 20b are made by applying pressing, deposition and other similar techniques to the substrate 10 of the support of the invention.

The material used for making the portions 20a, 20b is preferably the same, although different types or compositions may be used, preferably based on carbon and/or other elements.

It should be taken into account, as will be further explained hereinafter, that the use of carbon-based materials such as graphite, fullerene and other materials, will allow exploiting the heating effect due to the infrared emissions produced by such materials, in addition to the Joule effect.

In particular, the Applicant has attained favourable results by using, as a material for the electrodes 20 and their linear 20a and terminal 20b portions, carbon or compounds comprising carbon in its various forms available today for electric and/or electronic applications, more specifically its nanometric forms such as graphene, germanene, fullerene and nanotubes, and graphite as well.

Nanomaterials, in fact, especially composite polymeric ones obtained by adding graphene or nanotubes into the polymeric matrix, in addition to offering good electric conductivity properties are characterized by good thermal stability, flexibility, elasticity and tensile resistance (see, for example: Il grafene: proprietà, tecniche di preparazione ed applicazioni [Graphene: properties, preparation techniques and applications]—publ. Energia, Ambiente e Innovazione, no. 3-2011, ENEA journal). This will allow them to adhere potentially well to the base layer 10 and to adapt to any deformation or bending that the latter may undergo during the transdermal delivery treatments.

According to a preferred embodiment of the invention, the base layer 10 is made of or coated at least partly with a material that can combine with carbon to form therewith a coherent structure; this will affect the application techniques that will be used.

For example, CVD (chemical vapor deposition) application techniques require high temperatures, and therefore the base layer 10 will need to have appropriate heat-resistance properties. In this case, silicone-based materials capable of withstanding temperatures in excess of 100° C. will be preferable.

As far as graphene is concerned, on the other hand, when it is obtained by mechanical and/or chemical exfoliation it can be made to adhere to the base layer by burying it into the polymeric matrix or by causing it to adhere to the polymeric matrix after heating the latter. In this case, a thermoformable material or coating will be selected for the base layer 10, or cold fixing techniques may be used.

When the carbon of the electrodes 20, inclusive of the portions 20a, 20b, is in graphite form, it can be applied by pressing or spraying. To this end, suitable inks for three-dimensional (3D) printers may also be used.

Products suitable for this purpose are commercially available (e.g. from company Graphene 3D, Calverton-USA).

The choice among the different technological options for the production of the supports 1 according to the invention will depend on several factors, including production numbers (large or small scale production), the shape and size of the electrodes 20, the planned costs of the support 1 to be manufactured, the material of the base layer, etc.

The thickness of the carbon-based material deposited onto the base layer 10 in order to create the electrodes 20 and the portions 20a, 20b may change as a function of the intended applications of the support 1.

In fact, the electrodes 20 and their parts 20a, 20b must be sized essentially according to the current intensity that they may have to conduct, which in turn will depend on the iontophoretic treatment to be carried out; for typical current values (a few milliamperes), layers of a few tenths of a millimeter (0.03-0.8) will be sufficient.

In the example considered herein, the electrodes 20 are associated with a multiple connection appendix 22, which extends from the support 1 so that it can be electrically connected to a conjugated external connector 25 through a plug-socket coupling.

For this purpose, the appendix 22 is rigid or semi-rigid, so that it can be used as a socket or a plug to be coupled to the external USB connector 25 of FIG. 5a, or it may be soft or flexible, so that it can be coupled (“crimped” in expert jargon) to an RGB connector 25′ like the one shown in FIG. 5b.

Preferably, the support 1 comprises a coating layer 30 containing the substance to be administered transdermally.

To this end, the substance is prepared in gel form, in which the active principles to be administered are dispersed. At room temperature, it essentially looks like an elastic and adhesive film, which on one side coats the base layer 10 and on the other side is intended to be applied onto the patient's skin.

The gel can be spread by lamination onto the base layer 10, with the electrodes 20 and their portions 20a, 20b already disposed thereon, or it may be applied by using any other appropriate technique (doctoring, spraying, immersion, etc.); in this respect, reference can be made to the prior art concerning the application of active principles (e.g. diclofenac) onto pain-killing and antirheumatic patches or the like. Preferably, the coating 30 is protected by a plastic film 31 (dashed in FIG. 3) to ensure hygienic conditions when the support 1 is handled for application to a patient; of course, the protective film 31 is removed before laying the support onto the patient's skin.

In this condition, the support 1 can be connected to the power supply unit, e.g. like the one described in WO 02/24274, at the connection appendix 22 of the electrodes 20.

As shown in FIG. 2, the portions 20a of the latter are close to one another, and this represents an advantage achieved by the invention, wherein the electrodes 20 comprise portions 20a that can be configured as conductors, the ends of which can be set close to one another and associated with the multiple connection appendix 22 to establish the electric connection between the support 1 and the power supply. In this manner, in fact, it is possible to connect all the electrodes simultaneously through an appropriate multiple connector 25, 25′, 25″, 25′″; advantageously, the connector is either a plug or a socket (i.e. either female or male), and preferably of the type commonly used for multiple connections, such as USB, RGB or the like, conjugated to the connection appendix 22 of the support.

The connector 25 comprises, therefore, a plurality of contacts 26, which in the example shown in FIG. 5a are incorporated into a USB socket and are coupled to the connection appendix 22 of the support 1; to this end, the connector 25 is electrically powered by a cable 27 connected upstream to the control unit (not shown in FIG. 5a).

In one possible alternative embodiment, the multiple connector 25′ can be partially opened as shown in FIG. 5b, in order to engage the contacts 26′ with the appendix 22 of the support 1.

The appendix 22 and the multiple connector 25, 25′ will allow an operator to quickly establish the electric connection between the control unit or the power supply unit and the electrodes 20a, 20b, as well as to remove such connection at the end of a patient's treatment session; this is done by simply coupling or decoupling the connector 25 and the appendix 22 of the electrodes, just like a normal USB key

In light of the above, it can be easily understood how the support 1 according to the invention can solve the technical problem addressed by the invention.

In fact, the electrodes of the support 1 are preferably powered in succession in a sequential manner, with a time offset of approx. one or a few seconds.

The electric charge and the resulting field are distributed in the linear part 20a and in the terminal part 20b, in such a way as to achieve in-depth absorption of the substances.

The support 1 of the invention operates as follows: when the electrodes 20, 20a, 20b are energized through a succession of electric stimulations consisting of a periodic wave type, which are repeated at a modulated frequency at intervals of approx. 1-5 seconds, the molecule of the active principle is allowed to penetrate the cutaneous barrier through the ionic channels of the cellular membrane, thereby immediately activating the metabolism thereof.

After a part of the body, e.g. a thigh, a shoulder, the chest, etc., has been enveloped with the support 1, or a part of the body has been placed between a pair of supports 1 and 100 like those shown in FIG. 8, with the respective electrodes having opposite signs, i.e. with n pairs of positive and negative (+ and −) electrodes, action is taken individually and sequentially by concentrating the current into the underlying tissue, so as to activate the intracellular metabolism of the molecule conveyed by part of the receiver tissue. This makes it possible to carry out proper topical-regional treatments (as opposed to systemic treatments, which enter the blood circulatory system), down to a depth of approx. 10 cm, depending on the frequency, without damaging the skin and other organs and without interacting with the circulatory system.

All this is obtained in a simple and effective manner through the use of a single multiple connector 25 or 25′ (or 25″ or 25″'), which allows all the electrodes 20 to be simultaneously connected, via the appendix 22, to the respective portions 20a, 20b on the support 1, and to the power supply unit; in this regard, it is worth pointing out that the connector 25 is connected upstream to the power supply unit, or it may also be incorporated therein (e.g. as a USB port).

Thus, an operator will find it easier to put the support 1 in operation on a person, since he/she will simply have to apply the connector 25 or 25′ to the support in order to connect all the electrodes 20 simultaneously to the power supply unit.

Therefore, there is no risk that a mistake might be made when making the connections, because the latter are pre-arranged by the connector; this results in a big improvement over the prior art.

This achievement is also made possible by the presence, on the base layer 10, of at least one linear portion 20a, in addition to the terminal one 20b, of the electrodes 20:

in fact, this allows providing contact ends 22 suitable for use with a connector 25, preferably, but not limited to, a USB connector.

In addition, it must also be considered that the use of a material like carbon for the electrodes allows obtaining a support having innovative properties compared to the prior art. For example, the complete elimination of all metallic parts will prevent allergy problems caused by contact with the skin, which problems affect prior-art supports.

Of course, the invention may be subject to variations with respect to the description provided so far.

Variants may concern both the general configuration of the support 1 and the configuration of the single components thereof, such as the base substrate 10 or the electrodes 20.

As far as the first type of variant is concerned, reference should be made to FIG. 8 (wherein the same reference numerals are used for the support as in the preceding figures, adding 100 to those relating to a second support 100), which shows an embodiment of the invention that comprises a pair of supports, designated by reference numerals 1 and 100, which are preferably, but not necessarily, symmetrical and comprise each a number of electrodes 20, 120 similar to those already described, i.e. with a linear connection portion 20a, 120a and a terminal portion 20b, 120b.

As previously explained, the electrodes 20, 120 of each support 1, 100 are preferably powered sequentially in pairs having electrically opposite signs, i.e. positive and negative, and vice versa.

As regards the second type of variant, FIGS. 4a, 4b, 4c show three different possible embodiments of the support 1, wherein the elements corresponding to those already described are designated by the same numerals with the addition of a respective apostrophe (′, ″ and ′″).

The supports 1′, 1″, 1′″ are configured for application onto different parts of the body: the first one is for the face, the second one is for the neck, and the third one is for the lumbar region. As a consequence, as can be seen, the electrodes 20′, 20″, 20′″ have different configurations of their linear portions 20a′, 20″ and terminal portions 20b′-20b′″.

An end 22′-22′″ of the electrodes 20′-20′″ allows connecting to a USB connector for power supply.

The connection appendix 22 may also differ according to the selected type of plug-socket coupling, just like the corresponding connector 25, 25′. For this purpose, FIGS. 6a, 6b, 6c show some respective possible variants (though other variants are still possible) of the configuration of the connector 25, 25′.

In this case as well, apostrophes are used in FIGS. 6a-6d to designate the elements while keeping the same numerals. FIG. 6a shows an RGB connector 25′, similar to that of FIG. 5b, in the closed condition.

FIGS. 6c, 6d show respective multiple connectors 25″ and 25′″ having different configurations as concerns both the contacts 26″, 26′″ and the conductors 27″, 27′″. However, the effect obtained is always to allow for easy and fast connection of a plurality of electrodes 20 of the support 1 to an external power supply unit.

Another variant is finally visible in FIG. 7, which concerns the layers of the support 1. In this case as well, the same numerals designate elements that are structurally or functionally similar to those previously described.

As can be seen, in this case between the base layer 10 and the electrodes 20 there is an intermediate layer 35 acting as a coating for the base layer to promote the fixing of the electrodes 20, in particular of the material they are made of, e.g. carbon.

The intermediate layer 35 may consist of a resin impregnating the base layer 10, or a film applied onto the latter by lamination, glueing, stitching or the like.

The teaching of the invention should be considered to include also the various possible shapes of the supports for transdermal application other than the simple substrate shown in the drawings; this is the case, for example, of supports configured as envelopes containing the substance to be applied (in gel form or the like).

In fact, it can be easily understood that electrodes 20, with their portions 20a, 20b, can be applied onto one or more walls of the an envelope for connecting to a multiple connector 25 through ends 22 according to a USB-type connection.

In this respect, it must be highlighted that the envelope does not need to be wholly flexible, since it will be sufficient that there is at least one flexible wall carrying the electrodes and intended to be put in contact with the skin of a person to whom the substances have to be administered.

Finally, FIG. 9 shows a further possible variant of a support according to the invention, designated by reference numeral 200, wherein the electrodes 220 comprise a longer linear portion 220a and a proportionally reduced terminal portion 220b.

These variants will still fall within the scope of the following claims.

1-18. (canceled)

19. A support for transdermal application of substances, comprising:
a base layer and at least one electrode applied onto the base layer, characterized in that the electrode comprises at least a substantially linear portion and a terminal portion joined together.

20. The support according to claim 19, wherein the linear portion and the terminal portion of the electrode are applied onto the base layer.

21. The support according to claim 19, wherein the ratio between the areas of the terminal portion and of the linear portion is in the range of approximately 1 to 2.5.

22. The support according to claim 19, wherein the substantially linear portion of said at least one electrode is associated with a connection appendix adapted to engage with external connector means for supplying power to the electrode.

23. The support according to claim 22, wherein the appendix extends from an edge of the base layer.

24. The support according to claim 19, comprising a plurality of electrodes provided with respective substantially linear portions associated with the connection appendix, so as to be suitable for multiple connection to external connector means.

25. The support according to claim 24, wherein the multiple connection appendix and/or the multiple connector means are of the USB or RGB type or the like.

26. The support according to claim 19, wherein said at least one electrode, with its linear portion and terminal portion, is made of a material comprising carbon.

27. The support according to claim 26, wherein the carbon is at least partly in an allotropic form selected from the group including graphene, fullerene, nanotubes, graphite.

28. The support according to claim 19, wherein the linear portion and the terminal portion of the electrode are applied onto the base layer by using at least one of the following techniques: chemical deposition, hot-pressing, cold-pressing, spraying.

29. The support according to claim 19, comprising a coating layer of the base layer and/or of the electrodes, which contains the substance to be administered transdermally.

30. The support according to claim 19, comprising an intermediate layer interposed between the base layer and the electrodes, to which the latter are applied.

31. The support according to claim 19, wherein the base layer is made of a material included in the following group: fabric, non-woven fabric, felt, hide, leather, and the like

32. The support according to claim 19, wherein the base layer is made out of a sheet of plastic material, such as polyester, polyvinyl chloride (PVC), polyurethane, polyethylene, silicone, latex, and the like.

33. The support according to claim 19, wherein the surface extension of the electrodes, inclusive of both the linear portion and the terminal portion, is preferably in the range of 0.5 to 3 cm².

34. An apparatus for transdermal application of substances, characterized in that it comprises at least one support according to claim 19, wherein the electrodes are powered sequentially, with a time offset in the range of approximately 1 to 5 seconds, the electrically positive and negative signs being preferably alternated at every impulse cycle.

35. The apparatus according to claim 34, wherein the electrodes are powered sequentially in pairs, with electrically positive and negative signs within a single pair.

36. The apparatus according to claim 34, comprising a pair of supports, the electrodes of which are powered sequentially in pairs, with electrically positive and negative signs alternated at every impulse cycle.
